(19) United States
(12) Patent Application Publication
(10) Pub. No.: US 2013/0338127 A1
(43) Pub. Date: Dec. 19, 2013

(54) TRIAMCINOLONE ACETONIDE FORMULATIONS FOR TREATING DERMATITIS AND PSORIASIS

(71) Applicants: Hyder Aliyar, Midland, MI (US); Jie Zhang, Salt Lake City, UT (US)

(72) Inventors: Hyder Aliyar, Midland, MI (US); Jie Zhang, Salt Lake City, UT (US)

(73) Assignee: Nuvo Research Inc., Mississauga (CA)

(21) Appl. No.: 13/695,669

(22) PCT Filed: May 4, 2011
(86) PCT No.: PCT/US2011/035216

§ 371 Date: May 30, 2013

Related U.S. Application Data
(60) Provisional application No. 61/332,157, filed on May 6, 2010.

(51) Int. Cl. A61K 31/58

(52) U.S. Cl. CPC A61K 31/58
USPC 514/174

(57) ABSTRACT

The present invention is drawn to formulations and related methods for treating dermatitis or psoriasis. The formulation can include triamcinolone acetonide, a polymer selected from the group of a poly(2-hydroxyalkylacrylate), a poly(2-hydroxyalkylmethacrylate), and combinations thereof. The formulation also includes a volatile solvent system including at least one volatile solvent, and a non-volatile solvent system including at least one non-volatile solvent.

### BACKGROUND

Dermatitis (also known as eczema) and psoriasis are common skin diseases. Dermatitis is particularly prevalent on the hands of workers in service industry because of the workers frequent contact with wet or irritating chemicals. Psoriasis is a common auto-immune skin disease. Both dermatitis and psoriasis can cause serious physical and/or psychological suffering to the patient regardless of the location on the body that these conditions occur, but they are particularly bothersome if they occur on the skin of the hand. Those afflicted with such disorders often have to use their hands in their work which can aggravate the condition.

A variety of drugs and delivery forms have been used in attempts to treat dermatitis and psoriasis. Unfortunately, current therapies have many drawbacks. Oral medications frequently have undesirable side effects. Topically applied ointments and gel medications can be unintentionally removed from the treatment area when the skin is contacted by other objects. Additionally, topically applied medications may spread onto surrounding healthy skin areas and cause undesirable effects including atrophy of unaffected healthy skin by exposure to corticosteroids. Accordingly, research continues to try to develop topical formulations that are easy to apply and maintain on the diseased skin area and that are capable of delivering the active drug for extended periods of time.

### DETAILED DESCRIPTION

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof.

In describing and claiming the present invention, the following terminology will be used.

The singular forms “a,” “an,” and “the” include plural referents unless the context clearly dictates otherwise.

The term “skin” as used herein is defined to include human skin located anywhere on the human body. In one aspect of the invention, the human skin can be the skin of the hands.

The phrase “effective amount,” “therapeutically effective amount,” or “therapeutically effective rates” of a drug refer to a non-toxic, but sufficient amount or delivery rates of the drug, to achieve therapeutic results in treating the condition for which the drug is being delivered. It is understood that various biological factors may affect the ability of a substance to perform its intended task. Therefore, an “effective amount”, “therapeutically effective amount” or “therapeutically effective rates” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a subjective decision. The determination of a therapeutically effective amount or delivery rates is well within the ordinary skill in the art of pharmaceutical sciences and medicine.

The phrases “diseased skin” or “treatment area” are defined as the skin region which is afflicted with dermatitis or psoriasis.

The term “sustained” when referring to drug delivery can be defined in terms of either in vitro permeability across human or hairless mouse skin or epidermis, or by a data collected from a pool of 12 or more human subjects, wherein the drop in mean drug delivery rate over a specified period of time (about 2 hours or longer) is not more than 50% from a peak drug delivery rate. Thus, compositions that are delivered over a sustained period of time include formulations that deliver a drug at substantially constant and therapeutically significant rates for an sustained period of time, e.g., about 2 hours, about 4 hours, about 8 hours, about 12 hours, about 24 hours, etc.

“Drug” or “the drug” refers to triamcinolone acetonide. The triamcinolone acetonide can make-up about 0.05 wt % to about 0.8 wt % of the formulations disclosed herein. In one embodiment, the triamcinolone acetonide can comprise about 0.1 to about 0.6 wt % of the formulation. In another embodiment, the triamcinolone acetonide can comprise about 0.5 wt % of the formulation.

“Volatile solvent system” can be a single volatile solvent or a mixture of volatile solvents, where all of the solvents (one or more) are more volatile than water or water per se. Similarly, the term “volatile solvent” includes water or individual solvents that are more volatile than water.

“Non-volatile solvent system” can be a single non-volatile solvent or mixture of non-volatile solvents, where all of the solvents (one or more) are less volatile than water. Similarly, the term “non-volatile solvent” includes individual solvents that are less volatile than water. The non-volatile solvents in the non-volatile solvent system substantially remain in the solidified layer after evaporation of substantially all or all of the volatile solvent system. Accordingly, the solidified layer formed in accordance with embodiments of the present invention typically includes the non-volatile solvent system that is formulated to provide therapeutically sufficient flux of the drug for a period of time sufficient to provide a therapeutic effect.

The term “sustained period of time” is defined as at least about 2 hours, at least about 4 hours, and often at least about 8 hours. In one embodiment, the sustained period of time can be at least about 2 hours to about 12 hours.

“Adhesive formulation” or “adhesive solidifying formulation” refers to compositions that have a viscosity suitable for application to a skin surface prior to evaporation of its volatile solvent system, and which can become a solidified layer after evaporation of at least a portion of the volatile solvent system. The application viscosity is typically more viscous than a water-like liquid, but less viscous than a soft solid. Thus, when a composition is said to have a viscosity “suitable for application” to a skin surface, this means the composition has a viscosity that is high enough so that the composition does not substantially run off the skin after being applied to skin, but also has a low enough viscosity so that it can be easily spread onto the skin. A viscosity range that meets this definition can range from about 100 cP to about 1,000,000 cP (centipoises), and often from about 1,000 cP to about 100,000 cP.

The term “drying time” refers to the time it takes for the formulation to form a non-messy solidified surface after application on skin under standard skin and ambient conditions, and with standard testing procedure.

“Solidified layer” describes the solid layer formed from an adhesive solidifying formulation after at least a portion of the volatile solvent system has evaporated and the formulation has formed a soft, coherent layer. The layer is capable of remaining adhered to the skin, and is often capable of maintaining good contact with the subject's skin for substantially the entire duration of application under normal skin and ambient conditions (and typically to most severities of diseased skin afflicted with dermatitis or psoriasis). It can be desirable that the solidified layer has enough strength so that it does not disintegrate into small pieces during intended application time and has acceptable adhesion with the skin so that it does not fall off the skin during intended application for most patients.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0 mm” should be interpreted to include not only the explicitly recited values of about 0.01 mm to about 2.0 mm, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. This same principle applies to ranges reciting only one numerical value. Furthermore, such an interpretation should apply regardless of the breadth of the range or the characteristics being described.

With the above definitions in mind, a formulation and related methods for treating dermatitis or psoriasis is provided. In one embodiment, a formulation for treating dermatitis or psoriasis includes triamcinolone acetonide, a polymer selected from the group of a poly(2-hydroxyalkylacrylate), a poly(2-hydroxyalkylmethacrylate), and combinations thereof. The formulation also includes a volatile solvent system including at least one volatile solvent, and a non-volatile solvent system including at least one non-volatile solvent.

In another embodiment, a formulation for treating dermatitis or psoriasis includes triamcinolone acetonide, oleyl alcohol, at least one of ethanol or isopropyl alcohol, and poly(2-hydroxyethylmethacrylate). In a further embodiment, a formulation for treating dermatitis or psoriasis includes about 0.05 wt % to about 0.5 wt % of triamcinolone acetonide, about 1.0 wt % to about 5 wt % oleyl alcohol, about 5.0 wt % to about 20 wt % of poly(2-hydroxyethylmethacrylate); and at least one of ethanol or isopropyl alcohol.

In an additional embodiment, a formulation for treating dermatitis or psoriasis can include triamcinolone acetonide, a polymer, a volatile solvent system including at least one C₂-C₃ alcohol, and a non-volatile solvent system including at least one non-volatile solvent. The polymer in the formulation has a higher solubility in a 1:1 w/w mixture of ethanol: water than in pure water or pure alcohol alone.

In an additional embodiment, a method of dermally treating dermatitis or psoriasis is provided. The method includes applying a formulation to a skin surface of a subject suffering from dermatitis or psoriasis, solidifying the formulation to form a solidified layer on the skin surface by at least partial evaporation of the volatile solvent system in the formulation, and maintaining the solidified layer on the skin surface such that the solidified formulation dermally delivers the triamcinolone acetonide at therapeutically effective rates for a period of at least 2 hours. The formulation used in the method includes triamcinolone acetonide, a solvent system including at least one volatile solvent and at least one non-volatile solvent, and a polymer selected from the group consisting of: poly(2-hydroxyalkylmethacrylate), poly(2-hydroxyalkylacrylate), and combinations thereof.

While the formulations, methods, and solidified layers of the current invention can be used to treat dermatitis (eczema) or psoriasis of skin areas anywhere on the human body, the special characteristics of the formulations and methods of the current invention are expected to be particularly beneficial for treating hand dermatitis and psoriasis.

The formulations of the present disclosure can have a viscosity suitable for application and adhesion to a skin surface prior to evaporation of the volatile solvent system. Additionally, the formulations, when applied to a skin surface can form forms a solidified layer after at least partial evaporation of the volatile solvent system, and can continue to deliver triamcinolone acetonide at the therapeutically effective rates after the volatile solvent system is at least substantially all evaporated.

The formulations of the present disclosure can include a volatile solvent or volatile solvent system. A number of volatile solvents and mixtures of volatile solvents can be used in the formulations disclosed herein. Non-limiting examples of volatile solvents that can be used in the formulations can include water, ethyl ether, methyl ether, denatured alcohol, methanol, ethanol, isopropyl alcohol, propanol, ethyl acetate, and mixtures thereof. These volatile solvents should be chosen to be compatible with the rest of the formulation. In one embodiment, the volatile solvent system can include ethanol. In another embodiment, the volatile solvent system can include isopropyl alcohol.

The volatile solvent system can comprise from about 40 wt % to about 80 wt % of the total formulation. In one embodiment, the volatile solvent system comprises about 50 wt % to about 70 wt % of the formulation. In yet another embodiment of the disclosure, the volatile solvent system can include a C₂-C₃ alcohol and the C₂-C₃ alcohol can make-up at least about 50 wt % to about 80 wt % of the total formulation. In another embodiment, the C₂-C₃ alcohol can make up about 52 wt % to about 70 wt % of the total formulation.

The non-volatile solvent system of the formulations of the present disclosure can be capable of facilitating the delivery of triamcinolone acetonide into the skin, particularly diseased skin, at therapeutically effective rates over a sustained period of time. The non-volatile solvent system can include one or more non-volatile solvents and should be compatible with the drug and the polymer of the formulation. Non-limiting examples of solvents which can be included in the non-volatile solvent system can include oleic acid, oleyl alcohol, polyethylene glycol (PEG), propylene glycol, butylene glycol, isopropyl myristate, glycerol, dipropylene glycol, dimethyl isosorbide, and mixtures thereof. In one embodiment, the non-volatile solvent system includes oleyl alcohol. In another embodiment, the non-volatile solvent system includes propylene glycol. Generally, the non-volatile solvent system can comprise from about 10 wt % to about 40 wt % of the total formulation. In one embodiment, the non-volatile solvent system can comprise about 15 wt % to about 30 wt % of the total formulation.

The non-volatile solvent system can be chosen or formulated to be compatible with the polymer, the drug, the volatile solvent system in the formulation. Similarly, the amount of non-volatile solvent system in the formulation should correlate to the amount and type of polymer used. In some embodiments, in order to obtain desired permeability for the drug and/or compatibility with the polymer of the formulation, or other ingredients, a mixture of two or more non-volatile solvents can be used to form the non-volatile solvent system. Because the non-volatile solvent system acts as the vehicle solvent for the delivery of the drug, it can be desirable that it alone is capable of facilitating the delivery of the drug at therapeutically effective rates over a desired, sustained period of time without the help from other ingredients in the formulation. The non-volatile solvent system can also serve as plasticizer of the polymer in the formulation, so that the solidified layer can be elastic and flexible.

The non-volatile solvent system and the polymer should be compatible with each other. Compatibility is defined as i) the solidifying agent does not substantially negatively influence the function of the non-volatile solvent system; ii) the solidifying agent can hold the non-volatile solvent system in the solidified layer so that substantially no non-volatile solvent oozes out of the layer, and/or iii) the solidified layer formed with the selected non-volatile solvent system and the solidifying agent has acceptable flexibility, rigidity, tensile strength, elasticity, and adhesiveness to skin. With this in mind, in one embodiment, the non-volatile solvent system should be present in the formulation such that it forms a non-volatile solvent system to polymer ratio of about 1:3 to about 4:1. In another embodiment, the non-volatile solvent should be present in an amount such that the formulation has a non-volatile solvent system to polymer ratio of about 1:2 to about 3:1. In yet a further embodiment, the non-volatile solvent should be present in an amount such that the formulation has a non-volatile solvent system to polymer ratio of about 1:1 to about 2.5:1.

The selection of the polymer can also be carried out in consideration of the other components present in the formulation. A factor in selecting the polymer is that the polymer should not significantly reduce the transdermal permeation driving force (as reflected in transdermal drug permeation flux). In other words, the transdermal permeability of triamcinolone acetonide produced by a solidified formulation should not be significantly lower than the flux produced by a solution of triamcinolone acetonide in the same non-volatile solvent present in the solidified formulation (the difference between the two is the net effect of the polymer).

In one embodiment of the present invention, the polymer can be a poly(2-hydroxyalkylacrylate), a poly(2-hydroxylalkylmethacrylate), or combinations thereof. Non-limiting examples of polymers that can be used include poly(2-hydroxyethyl acrylate), poly(2-hydroxypropyl acrylate), poly(2-hydroxybutyl acrylate), poly(2-hydroxyethyl methacrylate), poly(2-hydroxypropylmethacrylate) and poly(2-hydroxybutylmethacrylate), and combinations thereof. In one embodiment, the polymer can include poly(2-hydroxyethylmethacrylate). As shown in the examples, poly(2-hydroxyethylmethacrylate) allows for high transdermal flux of triamcinolone acetonide.

The molecular weights of the polymers used in the formulations disclosed herein can vary depending on the amount and type of polymer(s) used. Polymers with excessively high molecular weights can take extended periods of time to dissolve in the manufacturing process which can be undesirable. Polymers with excessively low molecular weights can result in excessive quantities being used in order to achieve the desired viscosity, e.g. a viscosity suitable for application to a skin surface without running off. Accordingly, in one embodiment, the polymers used in the formulations disclosed herein can have molecular weights of about 10,000 Daltons to about 10 million Daltons. In another embodiment, the polymers used in the formulations disclosed herein can have a molecular weight of about 100,000 Daltons to about 2 million Daltons. Generally, the polymer can make-up about 2 wt % to about 30 wt % of the total formulation. In another embodiment, the polymer can make-up about 5.0 wt % to about 20 wt % of the formulation.

In some embodiments, it may be desirable to add an additional agent or substance to the formulation so as to provide enhanced or increased adhesive characteristics. Non-limiting examples of substances which might be used as additional adhesion enhancing agents include copolymers of methylvinyl ether and maleic anhydride (Gantrez polymers), other acrylic acid based polymers such as carbomers and carbopols (e.g., Carbopol 981), polyethylene glycol and polyvinyl pyrrolidone, gelatin, low molecular weight polyisobutylene rubber, copolymer of acrylsan alkyl/octylacrylamido (Dermacryl 79), and various aliphatic and aromatic resins.

Chemicals capable of increasing the transdermal flux of triamcinolone acetonide can also be added to the formulation. Such chemicals are often referred to as permeation enhancers and examples of such compositions are known by those of ordinary skill in the art.

The formulations disclosed herein can be applied to a skin surface at a thickness that is able to provide therapeutic delivery of the drug. If the layer is too thin, the amount of the drug may not be sufficient to provide therapeutic effect or to provide for sustained delivery over the desired length of time. In one embodiment, the formulation can be applied to have a thickness of about 0.02 mm to about 0.5 mm. In another embodiment, the formulation can be applied to have a thickness of about 0.04 mm to about 0.2 mm.

The formulations of the present disclosure may be left of the skin surface for sustained periods of time in order to provide for sustained delivery of the drug. In one embodiment, the formulations can be formulated to form a solidified layer that can be maintained on the skin and deliver triamcinolone acetonide at therapeutically effective rates for a period of 2 hours to about 12 hours. In another embodiment, the formulations can form a solidified layer which is capable of being maintained on the skin and delivering triamcinolone acetonide at therapeutically effective rates for a period of at least 4 hours.

After delivery of the drug, the solidified layers formed by the formulations disclosed herein can be removed by any means known in the art such as by peeling or washing. In one embodiment, the solidified formulation layer can be washed off the skin after the desired drug delivery period using a solvent such as water, ethanol, or isopropyl alcohol, or solutions containing one or more of these compounds, such as those used in hand sanitizers.

### EXAMPLES

The following examples illustrate the embodiments of the invention that are presently best known. However, it is to be understood that the following are only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical embodiments of the invention.

### Example 1

### Dermal Flux of Triamcinolone Acetonide Formulations

A number of polymers were tested for use in the formulations in order to determine the effectiveness in the formulations of the current invention. A significant parameter in the tests was the transdermal drug permeation flux, or how much of triamcinolone acetonide can be delivered across a unit area of the skin per unit time (skin flux in the unit of (ng/cm²/h)).

Human cadaver skin (HMS) was used as the membrane for the in vitro flux studies described in herein. Prior to testing, the skin tissue was stored in the freezer. The epidermis layer removed from the skin tissue was mounted carefully between the donor and receiver chambers of a Franz diffusion cell. The receiver chamber was filled with pH 7.4 phosphate buffered saline (PBS). The experiment was initiated by placing the test formulation on the stratum corneum side of the skin sample. Franz cells were placed in a heating block maintained at 37° C. At predetermined time intervals, 800 μL aliquots were withdrawn and replaced with fresh PBS solution. Skin flux (ng/cm²/h) was determined from the steady-state slope of a plot of the cumulative amount of permeation versus time. A commercially available products containing 0.5 wt % triamcinolone acetonide were used as a control. The produces were either Triamcinolone Acetonide cream made by Fougera, containing 0.5% triamcinolone acetonide or 0.5% Triamcinolone Acetonide ointment USP distributed by Perrigo, MI 49010).

The formulations were prepared according to Table A.

TABLE A
Example Formulations

| | Formulations* | | | | | | | | | |
|---|---|---|---|---|---|---|---|---|---|---|
| Excipients (wt. %) | 1 | 2 | 3 | 4 | 5 | 6 | 7 | 8 | 9 | 10 |
| Polyurethane | 14.2 | — | — | — | — | — | — | — | — | — |
| Propylene glycol | 10.0 | 7.1 | 4.4 | 1.7 | 4.5 | — | 2.8 | — | 3.5 | 32.8 |
| Ethanol | 56.8 | 8.4 | — | — | — | — | 49.8 | 44.8 | 39.4 | 35.7 |
| Water | 14.2 | 41.4 | 20.9 | 4.3 | 19.8 | 9.9 | — | — | — | 9.8 |
| Oleyl alcohol | 4.3 | 4.1 | — | — | 7.0 | — | 0.3 | 2.5 | 1.6 | 3.9 |
| Polyvinyl alcohol | — | 20.7 | — | — | — | — | — | — | — | — |
| Glycerol | — | 15.9 | — | — | — | — | — | — | — | — |
| Gantrez 425 | — | 1.9 | — | — | — | — | — | — | — | — |
| S100 | — | — | 22.6 | — | 24.1 | 16.8 | — | — | — | — |
| Isopropyl alcohol | — | — | 29.8 | 42.2 | 29.2 | 36.6 | — | — | — | — |
| L100-55 | — | — | — | — | — | 7.1 | — | — | — | — |
| Tromethamine | — | — | 5.2 | — | 5.1 | 5.4 | — | — | — | — |
| Butylene glycol | — | — | 9.7 | — | 9.8 | 16.4 | — | 2.5 | — | — |
| E100 | — | — | — | — | — | — | 6.2 | — | — | — |
| Hexamethylene disiloxane | — | — | — | — | — | — | 40.3 | 44.8 | 47.2 | — |
| Ethocel EC-N7 | — | — | — | — | — | — | — | 5.0 | — | — |
| Isosteric acid | — | — | 6.9 | 7.1 | — | 7.3 | — | — | — | — |
| Triacetin | — | — | — | 8.8 | — | — | — | — | — | — |
| Cetyl alcohol | — | — | — | 2.8 | — | — | — | — | — | — |
| Span 20 | — | — | — | 4.3 | — | — | — | — | — | — |
| Plalstoid | — | — | — | 28.3 | — | — | — | — | — | — |
| VA-64 (PVA -Polyvinyl | — | — | — | — | — | — | — | — | 7.9 | — |
| acetate) | | | | | | | | | | |
| Carbopol-981 | — | — | — | — | — | — | — | — | — | — |
| Poly(2- | — | — | — | — | — | — | — | — | — | 17.4 |
| hydroxyethylmethacrylate) | | | | | | | | | | |

*All the formulations contain 0.5% (w/w) of Triamcinolone acetonide

As discussed above, the flux rate of each of the formulations was tested and measured. The flux rate of each of the formulations is set forth in Table B.

TABLE B
Flux rates

| | Polymers used | Flux (ng/cm²/h) | |
|---|---|---|---|
| Formulation | in the formulation | Formulation | Control |
| 1 | 1. PU (polyurethane) | 235.5 ± 148.4 | 6.5 ± 0 |
| 2 | 2. PVA (polyvinyl alcohol) | 1.4 ± 1.3 | 2.9 ± 3.8 |
| 3 | 3. S100 (methacrylic acid | 42.3 ± 37.6 | |
| | copolymer) | | |
| 4 | 4. Plastoid (acrylic polymer) | 17.9 ± 9.9 | |
| 5 | 5. S100 | 410.9 ± 116.5 | 2.3 ± 1 |
| 6 | 6. S100 + L100 (methacrylic | 427.1 ± 102.2 | 2 ± 1.5 |
| | acid copolymer) | | |
| 7 | 7. E100 (Butylated | 8.1 ± 3.9 | 0 ± 0 |
| | methacrylic copolymer) | | |
| 8 | 8. Ethocel (Ethyl cellulose) | 23.4 ± 3.4 | |
| 9 | 9. PVA-Polyvinyl Acetate | 17.8 ± 25.2 | |
| | (VA-64) | | |
| 10 | 10. Poly(2- | 1139 ± 117.9 | 1.3 ± 2.3 |
| | hydroxyethylmethacrylate) | | |

As is shown in Table B, the flux value obtained for the formulation containing a polyalkylmethacrylate, namely poly(2-hydroxyethylmethacrylate), was significantly higher than that of the other formulations.

### Example 2

### Triamcinolone Acetonide Containing Formulation

A formulation containing triamcinolone acetonide and poly(2-hydroxyethylmethacrylate) is prepared according to Table C.

TABLE C

| Ingredient | Percentage (w/w) |
|---|---|
| Poly(2-hydroxyethylmethacrylate) | 12.46 |
| MW = 1 million Daltons | |
| Carbopol 981 | 0.98 |
| Propylene glycol | 22.40 |
| Oleyl alcohol | 2.51 |
| Water | 4.57 |
| Ethanol | 56.58 |
| Triamcinolone acetonide | 0.50 |

### Example 3

### Triamcinolone Acetonide Containing Formulation

A formulation containing triamcinolone acetonide is prepared according to Table D.

TABLE D

| Ingredient | Percentage (w/w) |
|---|---|
| Poly(2-hydroxyethylacrylate) MW = 2 | 5.0 |
| million Daltons | |
| Carbopol 981 | 0.5 |
| Butylene Glycol | 13.84 |
| Oleyl alcohol | 4.92 |
| Water | 4.74 |
| Isopropyl Alcohol | 70.5 |
| Triamcinolone acetonide | 0.50 |

### Example 4

### Triamcinolone Acetonide Containing Formulation

A formulation containing triamcinolone acetonide is prepared according to Table E.

TABLE E

| Ingredient | Percentage (w/w) |
|---|---|
| Poly(2-hydroxypropylmethacrylate) | 19.9 |
| MW = 100,000 Daltons | |
| Propylene glycol | 14.7 |
| Oleyl alcohol | 3.8 |
| Water | 8.6 |
| Ethanol | 52.5 |
| Triamcinolone acetonide | 0.50 |

While the invention has been described with reference to certain embodiments, those skilled in the art will appreciate that various modifications, changes, omissions, and substitutions can be made without departing from the spirit of the invention. It is therefore intended that the invention be limited only by the scope of the appended claims.

1. A formulation for treating dermatitis or psoriasis, comprising:
a) triamcinolone acetonide;
b) a polymer selected from the group of a poly(2-hydroxyalkylacrylate), a poly(2-hydroxyalkylmethacrylate), and combinations thereof;
c) a volatile solvent system including at least one volatile solvent, and
d) a non-volatile solvent system including at least one non-volatile solvent.

2. The formulation of claim 1, wherein the formulation has a viscosity suitable for application and adhesion to a skin surface prior to evaporation of the volatile solvent system, wherein the formulation applied to the skin surface forms a solidified layer after at least partial evaporation of the volatile solvent system, and wherein said triamcinolone acetonide continues to be delivered at the therapeutically effective rate after the volatile solvent system is at least substantially all evaporated.

3. The formulation of claim 1, wherein the volatile solvent system comprises ethanol or isopropyl alcohol.

4-6. (canceled)

7. The formulation of claim 1, wherein the non-volatile solvent system includes a solvent selected from the group consisting of: oleic acid, oleyl alcohol, polyethylene glycol (PEG), propylene glycol, butylene glycol, isopropyl myristate, glycerol, dipropylene glycol, dimethyl isosorbide, and mixtures thereof.

8-9. (canceled)

10. The formulation of claim 1, wherein the polymer includes a poly(2-hydroxyalkylacrylate) or a poly(2-hydroxyalkylmethacrylate).

11. (canceled)

12. The formulation of claim 1, wherein the polymer is selected from the group consisting of: poly(2-hydroxyethyl acrylate), poly(2-hydroxypropyl acrylate, poly(2-hydroxybutyl acrylate, poly(2-hydroxyethylmetacrylate), poly(2-hydroxypropylmethacrylate), poly(2-hydroxybutylmethacrylate), and combinations thereof.

13. The formulation of claim 1, wherein the polymer includes poly(2-hydroxyethylmethacrylate).

14. The formulation of claim 1, wherein the polymer comprises about 2.0 wt % to about 30 wt %.

15. (canceled)

16. The formulation of claim 1, wherein the non-volatile solvent system comprises about 10 wt % to about 40 wt % of the formulation.

17. (canceled)

18. The formulation of claim 1, wherein the volatile solvent system comprises about 40 wt % to about 80 wt % of the formulation.

19. (canceled)

20. The formulation of claim 1, wherein the volatile solvent system includes a C₂-C₃ alcohol, and the C₂-C₃ alcohol comprises at least about 50 wt % of the formulation.

21. The formulation of claim 1, wherein the formulation has a nonvolatile solvent system to polymer ratio of about 1:3 to about 4:1.

22-23. (canceled)

24. The formulation of claim 1, wherein the triamcinolone acetonide comprises about 0.05 wt % to about 0.8 wt % of the formulation.

25. (canceled)

26. A method of dermally treating dermatitis or psoriasis, comprising:
a) applying a formulation to a skin surface of a subject suffering from dermatitis or psoriasis, said formulation comprising:
i) triamcinolone acetonide,
ii) a solvent system including at least one volatile solvent and at least one non-volatile solvent, and
iii) a polymer including at least one polymer selected from the group consisting of: poly(2-hydroxyalkylmethacrylate), poly(2-hydroxyalkylacrylate), and combinations thereof;
b) solidifying the formulation to form a solidified layer on the skin surface by at least partial evaporation of the volatile solvent system; and
c) maintaining the solidified layer on the skin surface such that the solidified formulation dermally delivers the triamcinolone acetonide at therapeutically effective rates for a period of at least 2 hours.

27. The method of claim 26, wherein the skin surface is a skin surface on the hand.

28. The method of claim 26, wherein the formulation is applied at a thickness of about 0.02 mm to about 0.5 mm.

29. (canceled)

30. The method of claim 26, wherein the solidified layer is maintained on the skin and delivers triamcinolone acetonide at therapeutically effective rates for a period of 2 hours to about 12 hours.

31. The method of claim 26, wherein the solidified layer is maintained on the skin and delivers triamcinolone acetonide at therapeutically effective rates for a period of at least 4 hours.

32. The method of claim 26, further comprising the step of removing the solidified layer by washing with a solvent, wherein the solvent is selected from the group consisting of ethanol, isopropyl alcohol, water, and combinations thereof.

33. (canceled)

34. A formulation for treating dermatitis or psoriasis comprising:
a) triamcinolone acetonide;
b) oleyl alcohol;
c) at least one of ethanol or isopropyl alcohol; and
d) poly(2-hydroxyethylmethacrylate).

35. The formulation of claim 20 for treating dermatitis or psoriasis comprising:
a) about 0.05 wt % to about 0.5 wt % triamcinolone acetonide;
b) about 1.0 wt % to about 5 wt % oleyl alcohol; and
c) about 5.0 wt % to about 20 wt % of poly(2-hydroxyethylmethacrylate).

36. A formulation for treating dermatitis or psoriasis, comprising:
a) triamcinolone acetonide
b) a polymer
c) a volatile solvent system including at least one C2-C3 alcohol, and
d) a non-volatile solvent system including at least one non-volatile solvent, wherein the polymer has a higher solubility in a 1:1 w/w mixture of ethanol:water than in pure water or pure alcohol alone.

37. formulation as in claim 36, wherein alcohol is selected from the group consisting of ethanol, isopropyl alcohol, 1-propanol, and mixtures thereof.

38. A formulation as in claim 36, wherein the polymer is selected from the group consisting of poly(2-hydroxyethyl acrylate), poly(2-hydroxypropyl acrylate, poly(2-hydroxybutyl acrylate, poly(2-hydroxyethylmetacrylate), poly(2-hydroxypropylmethacrylate), poly(2-hydroxybutylmethacrylate), and combinations thereof.
